(19) United States
(12) Patent Application Publication
(10) Pub. No.: US 2008/0241940 A1
(43) Pub. Date: Oct. 2, 2008

(54) FECAL SAMPLING DEVICE AND METHOD

(76) Inventor: Vincent P. LaStella, Clark, NJ (US)

Correspondence Address: STROOCK & STROOCK & LAVAN LLP, 180 MAIDEN LANE, NEW YORK, NY, 10038, US

(21) Appl. No.: 12/135,869

(22) Filed: Jun. 9, 2008

Related U.S. Application Data
(63) Continuation-in-part of application No. 10/896,607, filed on Jul. 22, 2004.

(51) Int. Cl. G01N 33/72; B01J 19/00

(52) U.S. Cl. 436/66; 422/68.1

(57) ABSTRACT

A specimen sampling device according to one embodiment has a first panel, a second panel, a sheet between the first and second panels for receiving the collected samples and a movable area creating metering apertures through which samples are received. The movable area is movable from a collection position, in which it is adjacent the sheet, to a test position, in which it is spaced away from the sheet. A method of obtaining a fecal sample is also disclosed.

### CROSS REFERENCE TO RELATED APPLICATIONS

This application is a continuation-in-part of U.S. application Ser. No. 10/896,607, filed Jul. 22, 2004, entitled FECAL OCCULT BLOOD TESTING DEVICE AND METHOD, which is incorporated herein by reference in its entirety.

### BACKGROUND OF THE INVENTION

1. Field of the Invention

The present invention relates, generally, to specimen collection and more particularly, to a method and device for collecting and determining the presence of occult blood in fecal matter and a test kit containing such a device.

2. Description of Related Art

Over 100,000 persons per year in the United States are afflicted with cancer of the colon and rectum. When the number of colon/rectal cancers occurring each year is combined with the number of cancers occurring in other digestive organs, including the esophagus and stomach, such cancers of the digestive system account for more occurrences of cancer than any other single form of the disease. Contrary to many other forms of cancer, early diagnosis and treatment of digestive tract cancer does result in a cure rate of 80% to 90%. If, however, the disease is not detected until the later stages, the cure rate drops significantly. Thus, early detection of the disease is important to successful treatment of digestive tract cancer.

Most, but not all, cancers of the digestive tract bleed to a certain extent. This blood is deposited on and in fecal matter excreted from the digestive system. The presence of blood in fecal matter is not normally detected, however, until gross bleeding, that is, blood visible to the naked eye, occurs. Gross bleeding, however, is symptomatic of advanced cancers.

Digestive tract cancers in the early stages, including pre-cancerous polyps, also tend to bleed, giving rise to occult (hidden) blood in the fecal matter. Other pathological conditions, such as Crohn's disease and diverticulitis, can also give rise to the presence of occult blood in the fecal matter.

It is known that because of the relatively high fat content of fecal matter, blood, when present, is not distributed uniformly throughout it. For this reason, obtaining multiple samples from different areas of each bowel movement is desirable; but even a single positive test from any part of the feces should be considered a positive result.

Accordingly, test equipment and test procedures have been developed for use by physicians in testing for the presence of occult blood in fecal matter. One of the most successful tests is manufactured and sold by SmithKline Diagnostics of Sunnyvale, Calif. under the trademark HEMOCCULT and disclosed in Pagano U.S. Pat. No. 3,996,006, which is incorporated herein by reference in its entirety. In general, the Pagano test employs an absorbent paper impregnated with a guaiac-reagent and encased in a special test slide having openable flaps on both sides of the test slide. To use the Pagano test slide, a sample of fecal matter is smeared onto the guaiac impregnated paper by opening the panel on one side of the test slide. Thereafter, the panel is closed. The panel on the opposite side of the test slide is then opened and a nonaqueous developing solution is applied to the guaiac-impregnated paper. If occult blood is present in the fecal matter smeared on the opposite side of the paper, the guaiac reaction will dye the paper blue, providing a positive indication of the presence of blood in the fecal matter.

One drawback of such devices is that the positive indication is often difficult to observe due to too much specimen being applied to the paper. Thus, a need exists for an improved fecal sampling device, including one having improved readability.

### SUMMARY OF THE INVENTION

Embodiments of the present invention satisfy the foregoing, as well as other, needs. In accordance with one embodiment of the present invention, there is provided a specimen testing device comprising: a first panel, a movable area forming a plurality of metering apertures for receiving a specimen therethrough, a second panel, and a sheet disposed between the first and second panels. The sheet preferably includes a test area aligned with the plurality of metering apertures such that depositing the specimen through the plurality of metering apertures deposits the specimen on the test area. The movable area is movable from a first position, in which it is adjacent the sheet for receiving the specimen and a second position, in which it is spaced away from the sheet, for reading the results of the test. Additionally, the sheet is preferably impregnated with a reagent, such as guaiac, for possible reaction to the specimen as part of a test procedure.

In accordance with another embodiment of the present invention, there is provided a method of testing for the presence of blood in fecal matter.

In yet another embodiment of the present invention there is provided a test kit including a specimen testing device described above and one or more reagents for performing a test on specimens.

### BRIEF DESCRIPTION OF THE DRAWINGS

The invention will now be described with reference to the accompanying drawings, in which:

FIG. 1 is a front perspective view of a device according to one embodiment of the invention showing three testing areas with three covers in the open position;

FIG. 2 is a rear perspective view of the device of FIG. 1;

FIG. 3 is a perspective view of an absorbent sheet according to one embodiment of the present invention;

FIG. 4 is a perspective view of the device of FIGS. 1 and 2 in an un-assembled state;

FIG. 5 is a perspective view of an alternate embodiments of the present invention;

FIG. 6 is a perspective view of an alternate embodiments of the present invention; and

FIG. 7 is a perspective view of yet another alternate embodiment of the present invention.

### DETAILED DESCRIPTION

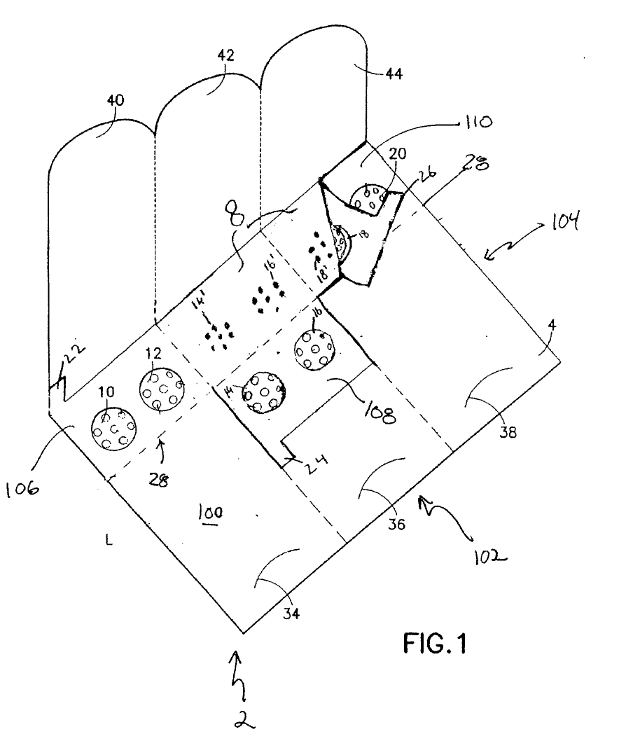

Referring to FIGS. 1-4, a fecal occult blood testing device 2 according to one embodiment of the invention, is shown. The device 2 generally includes three test panels 100, 102, 104 (although any number may be provided on a separate device) and is formed of a first panel 4 and a second panel 6, with an absorbent sheet 8 disposed between the first and second panels 4, 6, on which a specimen is placed.

Each test panel 100, 102, 104 is configured to receive samples through apertures in the first panel 4 and into the test areas 8′ of sheet 8. After receiving the specimen, the cover(s) 40, 42, 44 are closed, and the device 2 is turned over. Opening a flap 74, 76, 78 on the rear exterior of the device 2 (i.e., the second panel 6) exposes the test areas 8′ of the sheet 8 on which the specimen was deposited. Developing solution is added via the apertures exposed by opening the flaps 74, 76, 78. In a typical guaiac test, a blue color denotes a positive test result.

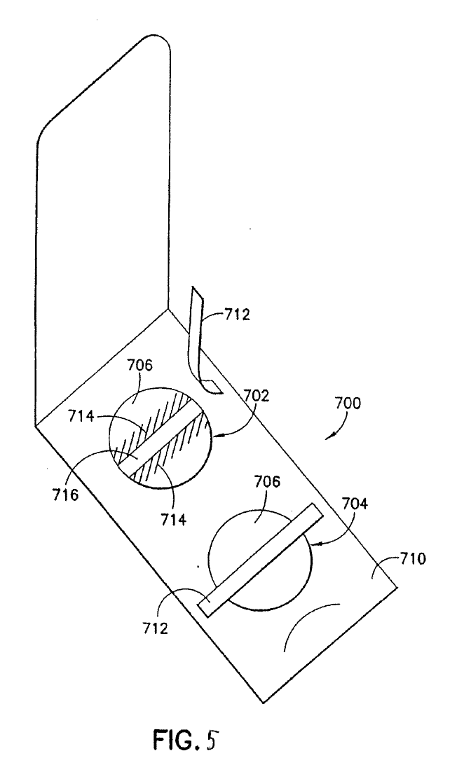

More specifically, the first test panel 100 includes two groups of metering apertures 10, 12 in panel 4 for receiving samples. Similarly, the second test panel 102 includes two groups of apertures 14, 16 for receiving samples, and the third test panel 104 includes two groups of apertures 18, 20 for receiving samples. The groups of apertures 10, 12, 14, 16, 18, 20 are formed by a movable area 106, 108, 110 of each test panel 102, 104, 106. It should be understood that the arrangement of the apertures (which may be one or more groups of apertures) 10, 12, 14, 16, 18, 20 for receiving samples can include, but are not limited to, an oval, circle, square, rectangle, linear series, and other shapes and arrangements, and each individual aperture may be any size or shape such as, for example square, rectangle, triangle, or circle. In further embodiments, the plurality of metering apertures can be a mesh, a permeable membrane, a woven material or other material. It should also be understood that the movable area may form the metering apertures in conjunction with other structures (such as the first panel, as in the embodiment of FIG. 5).

It should also be appreciated that, as compared to a single, large aperture, each plurality of smaller apertures 10, 12, 14, 16, 18, 20 preferably meters or controls the amount and configuration of the sample 14′, 16′, 18′ that gets deposited on the test areas 8′ of sheet 8 by receiving a portion of the sample and obscuring a portion of the sheet 8, thereby creating multiple spaced samples. Rather than a relatively large, thick sample, which can obscure the reading of the test results, the relatively smaller, spaced samples permit easier reading, as the developing solution, and thus the color, is able to permeate the space on sheet 8 between samples. The present inventor has discovered, however, that the readability of even the metered sample can be improved. It has been found that the portion of the movable area 106, 108, 110 surrounding the metering holes 10, 12, 14, 16, 18, and 20 can restrict or retard the developing solution, and thus the color test indicator, from permeating through the sheet 8 when the movable area is in contact (including partial contact) with the test sheet 8. Accordingly, the present embodiment includes movable areas 106, 108, 110, further discussed below.

As noted above, each test panel 100, 102, 104 includes a movable area 106, 108, 110 formed from the first panel 4. Each movable area 106, 108, 110 includes apertures 10, 12, 14, 16, 18, 20 formed therein. The movable area 106, 108, 110 may be defined by perforations, a crimp, fold or the like 28, or may be a separate piece coupled to the first panel 4 by tape or the like, thereby allowing for ease of folding, or spacing, each movable area 106, 108, 110 away from sheet 8 for testing. For example, as shown in FIG. 1 in connection with the second test panel 102, the movable area 108 has been folded along line 28 to overlay the first panel 4, thus placed in a test position. Alternatively, as also shown in FIG. 1 in connection with the third test panel 104, the movable area 110 can be placed in a test position by being removed from the device along line 28 and discarded. In FIG. 1, the movable area 106 of the first test panel 102 is in a collection position, adjacent sheet 8. In various other embodiments the movable areas may be removable tabs and the metering apertures may be created by (not only in) the tab, for example, as in the embodiment of FIG. 5.

To facilitate moving of each movable area 106, 108, 110, and to prevent contamination of the specimen and to promote sanitary conditions, grasping tabs 22, 24, 26 are provided. Preferably, the tabs 22, 24, 26 do not overlay or contact the specimen. In various embodiments, the tabs 22, 24, and 26 may be in any position related to test panels 100, 102, and 104 (e.g., extending as shown in FIG. 1, or perpendicular thereto, to the side). Furthermore, the movable areas 106, 108, and 110 may be any size or shape with relation to test panels 100, 102, and 104 so long as the functionality of the device as described herein is not impaired.

Each of the three panels 100, 102, 104 also has a cover 40, 42, 44, respectively. Each cover 40, 42 and 44 is engageable with a corresponding flap formed by arcuate slit 34, 36, 38, respectively, which is used to maintain the covers in a closed position, after the samples are obtained. Although not required, in the present embodiment, movable areas 106, 108, 110 are sized so as not to obscure slits 34, 36, 38 for securing the covers 40, 42, 44.

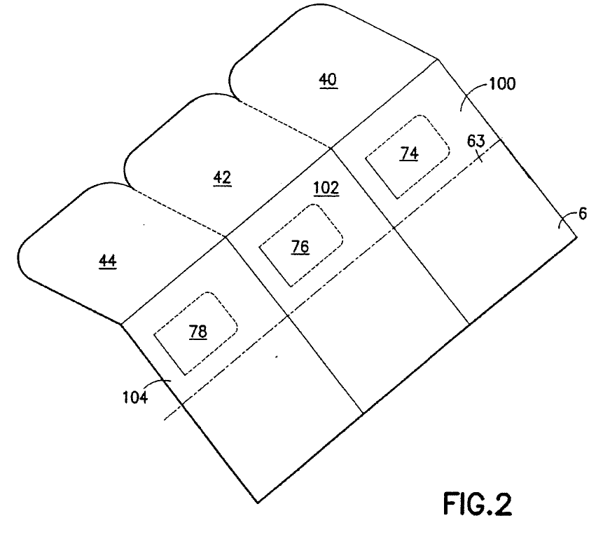

As shown in FIG. 2, each test panel 100, 102, 104 includes a flap 74, 76, 78 which is aligned with the metering apertures 10, 12, 14, 16, 18, 20 of the first panel 4 and thus, the test areas 8′ of sheet 8. For example, test panel 100 includes a flap 74 aligned with the plurality of metering apertures 10 and 12 in such a way that opening of flap 74 exposes the test areas 8′ of sheet 8, which is aligned with the plurality of metering apertures 10 and 12. As discussed below, the flaps 74, 76, 78 are opened to allow a developing solution to be placed on the test areas 8′ sheet 8 when performing a test.

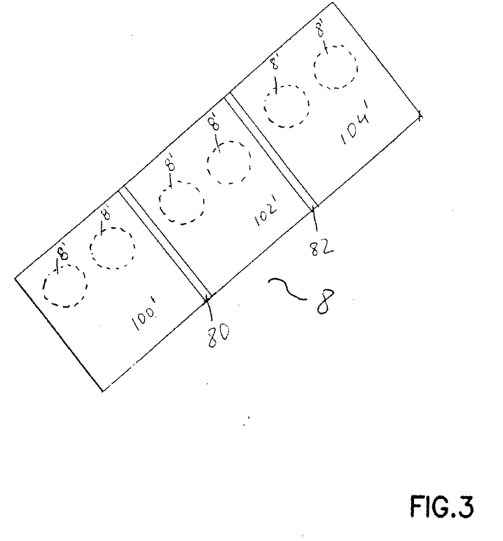

In the present embodiment, as illustrated in FIG. 3, the sheet 8 is a single piece of filter paper generally dimensioned to cover, and align with, at least the test apertures 10, 12, 14, 16, 18, 20 such that depositing a sample through the apertures 10, 12, 14, 16, 18 20 deposits the sample onto the test areas 8′ of sheet 8. In alternative embodiments, however, the sheet is dimensioned to cover the entire panel 6.

In certain embodiments, sheet 8 is made of an absorbent material, and is typically filter paper impregnated with a reagent which will react with hemoglobin components from blood and a peroxide developing solution to form a colored compound. Examples of suitable reagents are guaiac, tetraethyl benzidene, orthotoluidine and other similar chromogens, but any type of test may be used. In the embodiment illustrated herein, the reagent impregnated in sheet 8 is guaiac. Here, at least the test areas 8′ corresponding to the plurality of metering apertures 10, 12, 14, 16, 18 and 20 are impregnated with the reagent.

To reduce risk of cross-contamination, prevent or minimize possible leakage of developing solution and to ease separation of the three sheet panels 100′, 102′, 104′, the test panels 100, 102, 104 are separated from each other by dividing regions 80 and 82, which may comprise a hydrophobic material, for example wax, glue or other suitable material. Alternatively, the test area 8′ may be comprised of separate pieces of filter paper separated by a hydrophobic barrier. In still another embodiment, the samples are separated by a crimp or other physical barrier, for example, comprised of one or more of the panels 4, 6 themselves. As will be understood, the degree to which the sheet panels 100′, 102′, 104′ and the test areas 8′ are separated is dependent upon the tendency of the material used to manufacture the device to be affected by contamination. Furthermore, the device may comprise a single sheet panel.

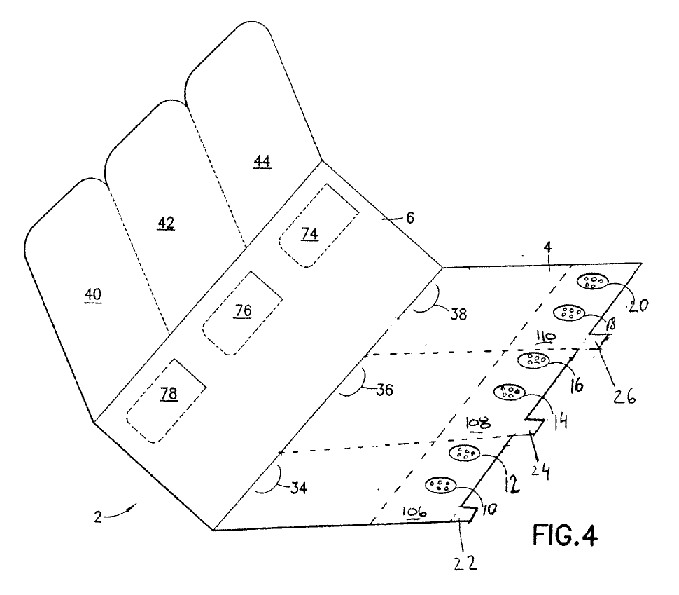

As illustrated in FIG. 4, the panels 4, 6 and covers 40, 42, 44, can be formed of a single sheet of paper, cardboard or other suitable material, in which the apertures, slits, tabs and perforations are die-cut. The device 2 is assembled by overlaying panel 4 on to panel 6 with the sheet 8 therebetween. The assembly is held together with a suitable glue or adhesive. Although not required, to minimize sticking of the covers to the specimen, the panels 4 and 6 are provided on their inner surfaces with a layer of non-stick material, typically a wax layer although other materials may be used.

The covers 40, 42 and 44 (where provided) for first panel 4 may be provided with appropriate printed matter to assist the patient, physician and/or laboratory. For example, the patient's name, address and instructions on how to use the device may be printed on the covers 40, 42 and 44. Such instructions may include instructing the patient to apply a specimen from the same areas of the fecal matter, or even the same smear, onto sample apertures 10, 12, 14, 18, 20. Other printed matter that may also be provided on the first panel 4 includes for example, the sample number and the test to be performed. Printed matter may also be provided on the second panel 6. For example, instructions to the doctor as to how to carry out testing by opening any flaps and/or tabs on second panel may be provided.

In a further embodiment, panel 4 can be provided with indicating means for locating where specimen is to be placed on the sheet. The indicating means may comprise printed circles or other shapes on the panel as a visible indicator to the user of where to place the specimen.

With regard to the embodiment of FIGS. 1-4, where a fecal sample is to be analyzed, the device 2 is typically sent home with a patient. The patient opens the cover 40 on the first panel 4 of the device and smears a first fecal specimen through metering apertures 10 on test panel 100, thereby depositing samples on the exposed test areas 8′ of sheet 8. In the present embodiment, a sample apertures are identified through markings on the first panel, namely, for receiving sample from the same area or region of the bowel movement being tested. A second fecal specimen, for example, taken at a different time as a result of a different bowel movement or from a different region of the same bowel movement as the first specimen, is then smeared through other corresponding metering apertures 12 of test panel 100, onto the exposed test areas 8′ of sheet 8. This may be performed any number of times, using any one or more test panels 100, 102, 104. With the samples taken, the patient may grasp the tab 22, 24, 26 and either fold or tear off the movable areas 106, 108, 110, thereby separating the test panels 100, 102, 104 having the metering apertures 10, 12, 14, 16, 18, 20 from sheet 8 having the samples. Then the patient preferably closes the covers 40, 42, 44 and secures them in the arcuate slits 34, 36, 38. Alternatively, the patient closes the covers 40, 42, 44 without moving the movable area(s) 106, 108, 110, which are left for the physician or laboratory to move when testing.

The patient obtains the requisite number of samples and typically either returns the device to the physician or to a laboratory.

To conduct the test or analysis on the specimens, the flap 74 on second panel 6 overlaying the apertures 10, 12 of the first test panel 100, through which the specimens have been applied, is opened and developer solution is applied to the exposed sheet 8. The testing can be repeated on the other test panels 102, 104 as needed. The spacing, accomplished by folding or removing movable areas 106, 108, and 110, in combination with the metering effect of the plurality of metering apertures 10, 12, 14, 16, 18, and 20 improves the ability to interpret the result of applying developer solution. In certain embodiments it is possible to apply the developing solution directly to the sample 14′, 16′, 18′, and 20′ through the front of the device 2 by opening the covers 40, 42, 44, thereby obviating the need for flaps 74, 76, 78 on the second panel 6.

In certain embodiments where the movable area includes one or more movable tabs, the tabs cover a portion of aperture(s) formed in the first panel. In such embodiments (as in FIGS. 5-7), it is possible to apply the developing solution in connection with the test directly to the front of the device, on the test area(s) of the sheet (although flaps on the second panel can be used). In such embodiments, the tab or tabs are moved or removed, thereby exposing areas of the sheet with and without the specimen. Because the tab or tabs provide a metering effect (as also shown in FIGS. 5-7), controlling the amount of specimen in the well and thus on the sheet, the physician or laboratory is able to read the results of the test without the need for a flap on the rear of the device. Even where a rear flap is used, the metering effect is desirable, as the test results are easier to read. The tabs obscure a portion of the wells and the sheet, thereby forming metering apertures. The tabs may be in the form of a strip, such as a metal, plastic, wax coated paper, impervious paper or other material. These tabs are movable, either by using releasable adhesive to attach it to the first panel or by using perforations other means allowing the tab to be torn away, folded away, or otherwise spaced away from the sheet.

Turning to FIG. 5, there is shown a device 700 having two wells 702, 704 for receiving samples. In general, the device 700 is constructed similar to the prior embodiments and includes a sheet 706 between a first panel 710 and a second panel (not shown). Apertures in the first panel 710 define the wells 702, 704. Each well 702, 704 is partially covered by a tab 712 in the form of a movable and here a removable tab 712 thereby forming two metering apertures in each of the wells 702 and 704.

In use, a patient smears a specimen in a well 702, thus causing the sample to be placed on the sheet 706 as well as on the tab 712. Accordingly, the well 702 includes areas both containing the specimen 714, as well as areas without the specimen 716.

When performing the test procedures, the physician or laboratory proceeds generally as described above, after removal of the tab 712, by opening a flap on the rear of the device and applying developing solution, or by applying the developing solution directly onto the sample through the aperture in the first panel 710. Tab 712 may be removed and discarded; however, it may also be used in connection a secondary testing procedure. Additionally, the tab 712 extends beyond the aperture, thereby providing a means for easy removal, without contamination of the sample.

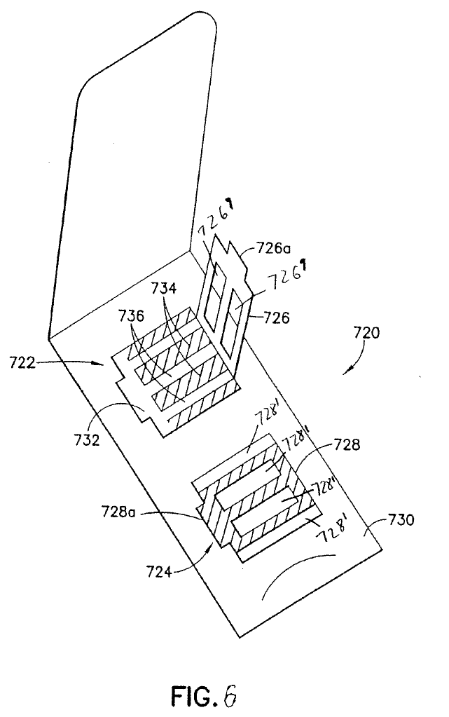

In the embodiment of FIG. 6, the device 720 similarly includes two wells 722, 724, each for receiving both primary and secondary samples. In the present embodiment, however, rather than using a tab formed of a strip adhered to the surface of the first panel, the present tabs 726, 728 are formed by punching out or otherwise forming portions of the first panel 730. Preferably, each tab 726, 728 forms metering apertures 726′, 728′ and includes an extension 726a, 728a or other portion extending away from the area receiving the specimen, thereby providing the tester an area for grasping the tab 726, 728 for easy folding and/or removal without contaminating the specimen.

As shown in connection with the first well 722, in which the tab 726 is in an open position, placing the sample in the well 722 results in a pattern on the sheet 732 of areas with specimen 734 and areas without the specimen 736. Again, a single smear of specimen results in both the specimen 734 on the sheet 732 and on the metering tab 726, which obscures or covers a portion of the sheet 732, thus resulting in a sample spaced on the sheet 732. As in prior embodiments, the metering portion—here the tab 726—is movable from a first position in which it is adjacent the sheet 732 for receiving the sample 734 and a second position, in which the tab 726 is spaced away from the sheet 732 for testing the sample 734.

In use, the test may be performed either by opening a flap on the rear of the device 720, and depositing the developing solution in the well 722, 724, either before or after removing the tab 726, 728 although readability is improved if the tab 726 is moved before or shortly after applying the developing solution. Alternatively, the tab 726, 728 can be opened and developing solution placed directly in the well 722, 724. Where developing solution is added after removal of tabs 726, 728 (so that tab 726 does not prevent the developing solution from permeating the sheet 732), the embodiment achieves the benefits noted above, namely a metering of the amount of specimen, as well as improved readability due to separation of the metering structure (here, tab 726, 728) from the deposited sample. Upon a positive test of the specimen, the corresponding tab 726, 728 may be used in connection with a secondary test to confirm a positive result or to identify a false positive result with a more specific test.

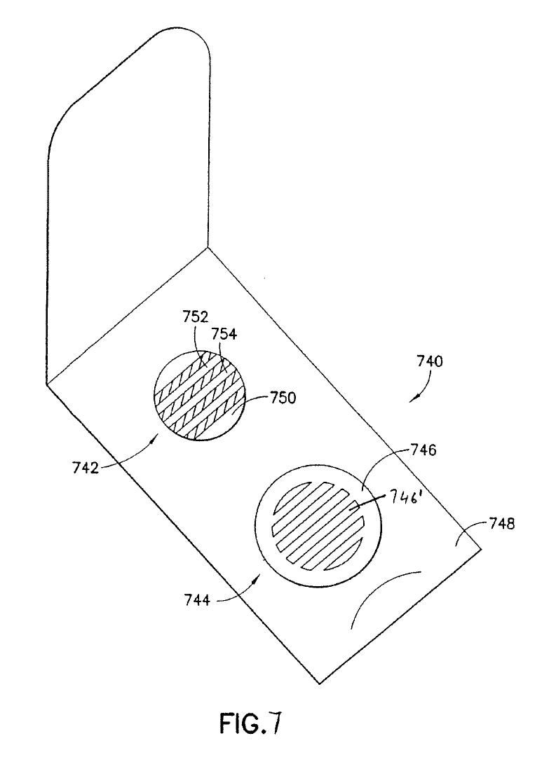

FIG. 7 illustrates another exemplary embodiment. The device 740 includes two wells 742, 744. Prior to use, each well 742, 744 is covered with a tab 746 with a plurality of openings 746′, which is adhered to the surface of the first panel 748 by removable or releasable adhesive. (Well 742 is shown after use, with the corresponding tab removed.) In the present embodiment, the tab 746 is wider than the well 744 to provide an area without specimen for grasping. In other embodiments, the tab can include an extension for ease of grasping.

In use, the patient smears the specimen in the wells 742, 744 across the tabs 746, thereby depositing some specimen in the well, on sheet 750 and some specimen on the tab 746. As shown in the first well 742, in which the tab has been removed, the tab, via metering apertures 746′, meters the amount of specimen in the well 742, thus resulting in a pattern of areas both with 752 and without 754 specimen.

As with the embodiments of FIGS. 5 and 6, the test procedure may be carried out via a flap on the back of the device 740 or by depositing developing solution directly into the well after removing the corresponding tab. Because the tab provides a metering function, the results of a the primary test may be read without the need for the flap on the rear and readability of the test results are improved by removal of the tab 746.

According to an additional embodiment, the testing device may be provided individually or may be packaged in kit form. For example, kits might be prepared comprising numerous testing devices, reagents required to perform the primary analysis for such devices, such as the developing solution used in the guaiac test.

Embodiments of the present invention enjoy numerous advantages. For example, the device can be embodied in one card that readily facilitates transference between the doctor and the patient and between the doctor and another testing location, such as a laboratory. The device is easy to use by the patient and is inexpensive to produce.

Those skilled in the art will recognize that the method and system of the present invention has many applications, may be implemented in many manners and, as such, is not to be limited by the foregoing exemplary embodiments and examples. In this regard, any number of the features of the different embodiments described herein may be combined into one single embodiment, and all features of a given embodiment need not be included in other embodiments. Moreover, the scope of the present invention covers conventionally known and future developed variations and modifications to the components and materials described herein, as would be understood by those skilled in the art.

Furthermore, although certain embodiments are shown with three test panels for testing three bowel movements, with multiple samples or specimens from each, other numbers of panels and/or samples/specimens may be provided and taken.

The embodiments of the invention has been described with reference to analysis of fecal samples for stool occult blood. However, the device may be used for screening and testing of other biological specimens, for example blood and AIDS tests, urine tests and pregnancy tests.

While the present invention has been described in considerable detail, the invention disclosed herein is not limited to the detailed description, and is to be afforded the full scope of the appended claims and all equivalents thereto.

What is claimed is:

1. A device for testing a specimen, the device comprising:
a first panel;
a movable area, the moveable area forming a plurality of metering apertures for receiving the specimen therethrough;
a second panel opposite the first panel; and
a sheet disposed between the first and second panels, the sheet including a test area aligned with the plurality of metering apertures,
wherein the movable area is movable from a collection position, in which the movable area is adjacent the sheet such that depositing the specimen through the plurality of metering apertures deposits the specimen on the test area, and a test position, in which the movable area is spaced away from the test area for testing the specimen.

2. The device of claim 1, wherein the movable area is removable from the device and when in the test position, the movable area is removed and separated from the device.

3. The device of claim 2, wherein the movable area is at least partially defined by a plurality of perforations in the first panel, the movable area being removable along the plurality of perforations.

4. The device of claim 1, wherein the movable area is at least partially defined by perforations in the first panel, the movable area being hingably movable along the perforations.

5. The device of claim 1, wherein the first panel and the second panel are formed from a single piece of material and are hingably connected to each other along a fold.

6. The device of claim 1, wherein the movable area includes a specimen-free grasping tab extending therefrom, the specimen-free grasping tab not aligned with any aperture for receiving specimen when the movable area is in the collection position.

7. The device of claim 1, wherein the second panel includes a flap, the flap covering an aperture in the second panel and adjacent the sheet aligned with the test area.

8. The device of claim 1, wherein the movable area is integrally formed as part of the first panel.

9. The device of claim 8, wherein the movable area has the plurality of metering apertures formed therein.

10. The device of claim 1, wherein the first panel includes an aperture aligned with the test area and the movable area is a tab releasably adhered to the first panel over a portion of the aperture.

11. The device of claim 10 wherein the aperture has an edge and metering apertures are formed between the tab and the edge of the aperture.

12. A method of collecting a fecal sample for testing for the presence of blood in fecal matter using a specimen testing device comprising a first panel, a movable area forming a plurality of metering apertures for receiving a specimen, a second panel opposite the first panel and a test sheet disposed between the first and second panels, the method comprising:
obtaining the device with the specimen deposited onto the test sheet when the movable area is in contact with the test sheet;
after the specimen is deposited through the metering apertures in the movable area, moving the movable area away from the test sheet;
applying developing solution to the test sheet; and
observing test results with the movable area spaced away from the test sheet.

13. The method of claim 12, wherein moving the movable area includes folding the movable area onto the first panel.

14. The method of claim 12, wherein moving the movable area includes removing the movable area from the first panel.

15. The method of claim 14, wherein the movable area is integrally formed from part of the first panel and the first panel includes a plurality of perforations defining the movable area and removing the movable area includes tearing the movable area away from the first panel along the perforations.

16. The method of claim 14, wherein the first panel includes a surface and the movable area is a tab releasably adhered to the surface of the first panel.

17. The method of claim 12, wherein the second panel includes a flap, the flap covering an aperture in the second panel and adjacent the sheet.

18. The method of claim 17, wherein applying developing solution to the test sheet includes adding the developing solution through the aperture in the second panel.

19. The method of claim 12, wherein applying developing solution to the test sheet includes adding the developing solution onto the side of the test sheet that was adjacent the movable area.

20. The method of claim 12, wherein the applying the developing solution to the test sheet occurs after the movable area is moved away from the test sheet.
